(19) United States
(12) Patent Application Publication
(10) Pub. No.: US 2013/0259947 A1
(43) Pub. Date: Oct. 3, 2013

(54) ORAL METRONIDAZOLE PHARMACEUTICAL COMPOSITIONS

(71) Applicants: Bijay Kumar Padhi, Orissa (IN); Muzammil Tariq, Okhla (IN); Sagar Dilip Mandawgade, Amravati (IN); Rajesh Gandhi, Madhya Pradesh (IN); Rajeev Singh Raghuvanshi, Gurgaon (IN); Dushyanth Surakanti, Somerset, NJ (US); Kent Allenby, Princeton, NJ (US)

(72) Inventors: Bijay Kumar Padhi, Orissa (IN); Muzammil Tariq, Okhla (IN); Sagar Dilip Mandawgade, Amravati (IN); Rajesh Gandhi, Madhya Pradesh (IN); Rajeev Singh Raghuvanshi, Gurgaon (IN); Dushyanth Surakanti, Somerset, NJ (US); Kent Allenby, Princeton, NJ (US)

(73) Assignee: DR. REDDY'S LABORATORIES LTD., Hyderabad, AP (IN)

(21) Appl. No.: 13/990,000

(22) PCT Filed: Nov. 29, 2011
(86) PCT No.: PCT/US2011/062406

§ 371 Date: Jun. 17, 2013

Related U.S. Application Data
(60) Provisional application No. 61/434,667, filed on Jan. 20, 2011.

(30) Foreign Application Priority Data
Nov. 29, 2010 (IN) 3592/CHE/2010

(51) Int. Cl. A61K 9/00; A61K 31/4164

(52) U.S. Cl. CPC A61K 9/0002; A61K 31/4164
USPC 424/494; 424/497; 514/398

(57) ABSTRACT

The present invention relates to an oral pharmaceutical composition comprising metronidazole, wherein metronidazole is released from the composition generally at the pH 5.0 and above.

### FIELD OF THE APPLICATION

The present application relates to an oral pharmaceutical composition comprising metronidazole, wherein metronidazole is released from the composition generally at a pH range of about 5.0 and above.

### BACKGROUND

Intestinal infections are common diseases caused by either foreign pathogenic pathogens of various origins colonizing the intestine, or normally present intestinal microorganisms becoming virulent.

The intestine is divided into two distinct portions, the “small intestine”, in the craniocaudal direction by the duodenum, the jejunum and the ileum, and the “large intestine”, formed by the colon and the recto-anus. The ileocaecal valve separates the small intestine from the large intestine. This valve permits the unidirectional passage of intestinal contents from the small intestine to the large intestine.

From a functional point of view, the large intestine is quite different from the small intestine. The small intestine's function includes food digestion and absorption, production of B-complex vitamins and vitamin K, metabolism of biliary acids and various other organic substances and to the rapid transfer of the alimentary bolus to the sections further downstream. The large intestine absorbs water, for the digestion of vegetable fibers and for the completion of some digestive processes initiated in the small intestine.

Unlike the small intestine, the large intestine contains rich bacterial flora, the balance of which is important in regulating the ambient pH, motility, producing gas and ammonia, forming faeces, and producing metabolites essential for maintaining appropriate function.

These characteristics are believed to be responsible in part to some of the debilitating pathologies which occur in the large intestine and in particular, the colon. For instance, the colon is host to a large number of bacterial strains. Further, it possesses varying levels of pH, anaerobiosis, humidity, and fecal transit time. These can contribute to disrupting existing flora which results in virulent or pathogenic bacteria colonizing and proliferating in the colon.

Nosocomial infections associated with Clostridium difficile (C. difficile), are known to result in endemic diarrhea. C. difficile is responsible for colitis in humans, antibiotic associated diarrhea and virtually all cases of pseudomembranous colitis. C. difficile associated diarrhea (CDAD) prolongs hospitalization, increases the costs of care, and causes considerable morbidity and mortality; both rates worsening in relapse cases. In particular, elderly patients and those who have long hospital stays exhibit high risk of relapse.

Currently available therapies for treating colon infections are believed to be insufficiently optimal, especially in the strong acidic environments, such as the gastric environment. Further, currently available therapy may not be effective due to intestinal biomass. Many patients respond to initial current therapy but then have recurrences of disease. Even more importantly, the current therapies may not inhibit cell wall synthesis or interrupt bacterial DNA synthesis. Finally, the current therapies are not always well-tolerated. Therefore, there is a long felt need to develop new therapies to treat a variety of colon infections.

Metronidazole is a nitroimidazole antibiotic with antibacterial activity against obligate anaerobic bacteria and certain protozoan parasites. It is the drug of choice for first episodes of mild-to-moderate C. difficile infections. The current branded therapy of metronidazole (FLAGYL® from G.D. Searle LLC) is an immediate release tablet formulation and it is available in either a 250 mg or 500 mg doses.

Although metronidazole is available in discrete dosage forms (FLAGYL®), it releases more rapidly thereby resulting in quick absorption through the digestive tract and reducing its availability within the colon lumen. This lack of intraluminal availability limits its utility to mild and moderate cases of CDAD. The second limitation of this active ingredient is that it could disrupts the normal colonic flora (i.e. bacteroids).

The usual oral antibacterial therapies for treating colon pathologies have often given contradictory results. For instance, excessive dilution of the active ingredient has been observed in the intestinal lumen. This dilution is believed to be due to the premature release of the antibacterial agent from the pharmaceutical form even before reaching the duodenum such as in the stomach and in the immediate vicinity of the patient's pyloric valve.

In order to enhance the desired therapeutic effects of the metronidazole, the inventors of the present patent application believe it to be necessary to control the metabolic degradation and drug distribution. A controlled and site-specific formulation reduces the likelihood of rapid degradation or distribution outside of the colonic lumen, thereby keeping intact the requisite efficacious dose.

The release of metronidazole in the terminal ileum/caecum leads to a much higher intraluminal concentration than that found with oral administration of an immediate release formulation.

A number of colon-specific formulations have been reported. These drug delivery systems are broadly categorized as follows: i) delayed release system designed to release drug with change in pH, ii) timed-release system designed to release drug after a predetermined time, iii) a microflora enzyme system using enterobacteria in the lower part of the gastrointestinal tract, and iv) a system using a lectin-like substance specific to the large intestine.

U.S. Pat. No. 5,290,559 discloses a controlled release unit of metronidazole ester comprising hydrogel. The rate of release of metronidazole is influenced by the presence of bacterial lipase, so that a higher concentration of infectious bacteria causes the release of higher concentrations of metronidazole.

U.S. Pat. No. 6,103,262 discloses metronidazole containing compositions which permit a slow release of metronidazole over a 24 hour period. The composition comprises different portions of granules containing metronidazole, capable of releasing metronidazole at a controlled rate over a 24 hour period.

U.S. Pat. No. 6,663,890 discloses a once-a-day metronidazole dosage form, which contains three types of dosage forms, first an immediate release dosage form; second and third delayed dosage forms. The three different dosage forms release metronidazole at different times, in which the Cₘₐₓ of the dosage form is achieved in less than about 12 hours.

It is challenging to design site specific compositions due to the high pH variability found during intestinal infections. Accordingly, there is a long felt need to develop a site specific metronidazole formulation specifically meant for the treatment of colon infections.

### SUMMARY

In one embodiment, the present application provides a metronidazole composition comprising:
(i) a core containing metronidazole, and
(ii) a release layer comprising at least one release material which dissolves/erodes in the aqueous environment at a pH of more than about 5.0.

In another embodiment, the present application provides an oral pharmaceutical composition of metronidazole that releases not more than 10% of metronidazole at a period of 120 minute in 900 ml of 0.1N HCl solution and not less than 60% of metronidazole at a period of 240 minutes in 900 ml of aqueous buffer having pH of 6.5, when tested in a USP Type 2 apparatus at 75 rpm and 37° C.

In another embodiment, the present application provides an oral pharmaceutical composition comprising:
(i) a core comprising metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5;
(iv) an optional protecting layer, wherein said composition has
   Cₘₐₓ of not more than 9.0 μg/mL,
   AUC₀₋₄₈ of not more than 120 μg·h/mL,
   Tₘₐₓ of not less than 4.0 hours, and
   T_{lag} of more than 1.0 hour:
   when administered to a human subject under fasting condition.

In another embodiment, there is provided a method of treating intestinal infections upon administration to a patient in need of treatment with metronidazole.

In another embodiment, the composition releases metronidazole substantially in the large intestine upon administration to a patient in need of treatment with metronidazole.

### DETAILED DESCRIPTION

The present application will be described in more detail below.

While the specification concludes with the claims particularly pointing and distinctly claiming the invention, it is believed that the present invention will be better understood from the following description. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having,” “including,” and “comprised of” are also to be construed as open ended unless the context suggests otherwise. All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

In one embodiment, the present application relates to an oral pharmaceutical composition comprising metronidazole, wherein metronidazole is released from the composition at a pH of greater than about 5.0.

In one aspect of the above embodiment, the present application provides an oral pharmaceutical composition comprising metronidazole, wherein metronidazole is released from the composition in the pH range of about 5.0 to about 8.0.

In another aspect, metronidazole is released from the composition in the pH range of about 5.5 to about 7.5.

In another embodiment, there is provided an oral pharmaceutical composition comprising:
(i) a core containing metronidazole, and
(ii) a release layer comprising at least one release material which dissolves/erodes in the aqueous environment at a pH of more than about 5.0.

In another embodiment, there is provided a metronidazole composition, wherein the release layer comprises at least one additional release material which dissolves in the aqueous environment at a pH of more than or equal to about 6.5.

In another embodiment, the composition of the present application optionally comprises a separating layer disposed between the metronidazole containing core and the release layer.

In another aspect of the above embodiments, the composition may comprise a protecting layer placed over the release layer.

In another embodiment, the thickness of the release layer ranges from about 60 μm to about 180 μm, or from about 70 μm to about 170 μm, or from about 80 μm to about 160 μm.

In another embodiment, the ratio of the two release materials forming the release layer is in the range of from about 05:95 to about 95:05, from about 10:90 to about 90:10, by weight. These release layers are capable of dissolving at different pH levels, i.e., at a pH more than or equal to about 5.0 and at a pH more than or equal to about 6.5 respectively.

In yet another embodiment, the ratio of core of the composition to the release layer ranges from about 4.0:1.0 to about 4.80:0.20, by weight.

In one embodiment, there is provided an oral pharmaceutical composition comprising:
(i) a core containing metronidazole,
(ii) an optional separating layer,
(iii) a release layer comprising at least one release material which dissolves at a pH more than or equal to about 5.0 and
(iv) an optional protecting layer, wherein the ratio of core of the composition to the release layer ranges from about 4.0:1.0 to about 4.80:0.20, by weight.

In another embodiment, there is provided an oral pharmaceutical composition comprising:
(i) a core containing metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5 and
(iv) an optional protecting layer, wherein the ratio of core of the composition to the release layer ranges from about 4.0:1.0 to about 4.80:0.20, by weight.

In another embodiment, the release layer of the composition comprises from about 1% to about 20% w/w, or from about 5% to about 15% w/w, or from about 2% to about 15% w/w of the composition.

In other embodiments, the composition of the present application additionally includes one or more pharmaceutically acceptable excipients or any combinations thereof. In particular, one or more excipients that are suitable for this composition are those which may act as diluents, binders, disintegrants, lubricants, glidants, surfactants, buffers, wetting agents, coloring agents, flavoring agents, etc.

In another embodiment, the present application provides an oral pharmaceutical composition that releases metronidazole, substantially in the large intestine upon administration to a patient in need of treatment with metronidazole.

In one aspect of the above embodiment, the compositions of the present application are useful in treating intestinal infections upon administration to a patient in need of treatment with metronidazole.

In one aspect of the above embodiment, the compositions of the present application are useful in treating infections of distal portion of small intestine or in the colon upon administration to a patient in need of treatment with metronidazole.

In another embodiment, the release material present in the release layer should generally be non-toxic and should be predominantly soluble in the intestinal fluid, but substantially insoluble in the gastric juices.

In another embodiment, the ratio of release materials forming the release layer comprise, poly(methacrylic acid-co-ethyl acrylate) {“EUDRAGIT® L 30 D-55”} and poly(methyl acrylate-co-methyl methacrylate-co-methacrylic acid) {“EUDRAGIT® FS 30 D”} in a ratio of about 05:95 to about 95:05.

In another embodiment, the core containing metronidazole may additionally comprise at least one rate-controlling agent.

In one aspect of the above embodiment, the rate-controlling agent present in the core amounts in the range of 0 to about 40%, or 0 to about 30%, or 0 to about 20% of the total weight of the composition.

In another embodiment, the present application provides an oral pharmaceutical composition comprising metronidazole, wherein said composition releases not more than 10% of metronidazole at a period of 120 minutes in 900 ml of 0.1N HCl, when tested in a USP Type 2 apparatus at 75 rpm and 37° C.

In another embodiment, the present application provides an oral pharmaceutical composition comprising metronidazole, wherein said composition releases not less than 60% of metronidazole at 240 minutes in 900 ml of pH 6.5 aqueous buffer, when tested in a USP Type 2 apparatus at 75 rpm and 37° C.

In another embodiment, the present application provides an oral pharmaceutical composition comprising metronidazole, wherein said composition releases not less than 70% of metronidazole at a period of 240 minutes in 900 ml of pH 6.5 aqueous buffer, when tested in a USP Type 2 apparatus at 75 rpm and 37° C.

In another embodiment, the present application provides an oral pharmaceutical composition of metronidazole, wherein said composition releases not more than 10% of metronidazole at a period of 120 minutes in 900 ml of 0.1N HCl solution and not less than 60% of metronidazole at 240 minutes in 900 ml of pH 6.5 aqueous buffer, when tested in a USP Type 2 apparatus at 75 rpm and 37° C.

In another embodiment, the present application provides an oral pharmaceutical composition comprising metronidazole, wherein said composition releases not more than 10% of metronidazole at 120 minutes in 900 ml of 0.1N HCl solution and not less than 70% of metronidazole at 240 minutes in 900 ml of pH 6.5 aqueous buffer, when tested in a USP Type 2 apparatus at 75 rpm and 37° C.

In one aspect of the above embodiments, wherein said composition having a release layer comprising at least one release material which dissolves/erodes in the aqueous environment at a pH of more than about 5.0.

In another aspect, wherein the release layer having at least one additional release material which dissolves in the aqueous environment at a pH of more than or equal to about 6.5.

In another embodiment, the present application provides an oral pharmaceutical composition comprising:
(i) a core containing metronidazole,
(ii) an optional separating layer,
(iii) a release layer comprising at least one release material which dissolves at a pH more than or equal to about 5.0, and
(iv) an optional protecting layer, wherein said composition releases not more than 10% of metronidazole at 120 minutes in 900 ml of 0.1N HCl solution, when tested in a USP Type 2 apparatus at 75 rpm and 37° C.

In another embodiment, the present application provides an oral pharmaceutical composition comprising:
(i) a core containing metronidazole,
(ii) an optional separating layer,
(iii) a release layer comprising at least one release material which dissolves at a pH more than or equal to about 5.0, and
(iv) an optional protecting layer, wherein said composition releases not less than 60% of metronidazole at 240 minutes in 900 ml of pH 6.5 aqueous buffer, when tested in a USP Type 2 apparatus at 75 rpm and 37° C.

In one aspect of the above embodiment, wherein the composition releases not less than 70% of metronidazole at 240 minutes in 900 ml of pH 6.5 aqueous buffer, when tested in a USP Type 2 apparatus at 75 rpm and 37° C.

In another embodiment, the present application provides an oral pharmaceutical composition comprising:
(i) a core containing metronidazole,
(ii) an optional separating layer,
(iii) a release layer comprising at least one release material which dissolves at a pH more than or equal to about 5.0, and,
(iv) an optional protecting layer, wherein said composition releases not more than 10% of metronidazole at 120 minutes in 900 ml of 0.1N HCl solution, and not less than 60% of metronidazole at 240 minutes in 900 ml of pH 6.5 aqueous buffer, when tested in a USP Type 2 apparatus at 75 rpm and 37° C.

In one aspect of the above embodiment, wherein the composition releases not less than 70% of metronidazole at 240 minutes in 900 ml of pH 6.5 aqueous buffer, when tested in a USP Type 2 apparatus at 75 rpm and 37° C.

In another embodiment, the present application provides an oral pharmaceutical composition comprising:
(i) a core containing metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5, and
(iv) an optional protecting layer,
wherein said composition releases not more than 10% of metronidazole at 120 minutes in 900 ml of 0.1N HCl solution, when tested in a USP Type 2 apparatus at 75 rpm and 37° C.

In another embodiment, the present application provides an oral pharmaceutical composition comprising:
(i) a core containing metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5, and
(iv) an optional protecting layer,
wherein said composition releases not less than 60% of metronidazole at 240 minutes in 900 ml of pH 6.5 aqueous buffer, when tested in a USP Type 2 apparatus at 75 rpm and 37° C.

In one aspect of the above embodiment, wherein the composition releases not less than 70% of metronidazole at 240 minutes in 900 ml of pH 6.5 aqueous buffer, when tested in a USP Type 2 apparatus at 75 rpm and 37° C.

In another embodiment, the present application provides an oral pharmaceutical composition comprising:
(i) a core containing metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5, and
(iv) an optional protecting layer,
wherein said composition releases not more than 10% of metronidazole at 120 minutes in 900 ml of 0.1N HCl solution and not less than 60% of metronidazole at 240 minutes in 900 ml of pH 6.5 aqueous buffer, when tested in a USP Type 2 apparatus at 75 rpm and 37° C.

In one aspect of the above embodiment, wherein the composition releases not less than 70% of metronidazole at 240 minutes in 900 ml of pH 6.5 aqueous buffer, when tested in a USP Type 2 apparatus at 75 rpm and 37° C.

In another aspect, the amount of said first pH dependent release material and the amount of said second pH dependent release material in a release layer are selected to target the release of metronidazole to the distal part of the small intestine or the colon of a patient in need of administration of metronidazole.

In another aspect, the present application provides an oral pharmaceutical composition comprising metronidazole, wherein the composition is designed in such a manner as to release the metronidazole substantially in the large intestine where the specific action of metronidazole is required, but leaving unaltered the non-pathogenic bacteria flora present in the small intestine which are not affected by the infection.

It has been surprisingly found that the composition containing a release layer or a release layer containing combination of different release materials, i.e. capable of dissolving at varying pH, complements the release mechanism and overcomes the limitations associated with currently available single-trigger systems. The release layer of the present invention dissolves within the pH range from about 5.0 to about 8.0, i.e. between the two extreme values representing highly variable patient groups. Thus, in one aspect, the release of metronidazole may start in the duodenum in patients with extremely ‘high’ pH profiles and proceed to the colon. Whereas, for patients with extremely ‘low’ pH profiles, the release of metronidazole would start in the distal part of the small intestine and further proceed to the colon.

In one aspect, the release layer of the present application dissolves/erodes within the pH range of from about 5.0 to about 8.0.

In one aspect, the release layer of the present application dissolves/erodes within the pH range of about 5.5 to about 7.5.

In one aspect, the oral metronidazole composition of the present application is capable of releasing metronidazole solely in the colon, thus ensuring localized antibacterial activity. Advantageously the site-specific delivery in the large intestine, particularly the colon, permits a greater concentration of metronidazole to be delivered to the site where it is most needed while preserving the healthy proximal regions.

In one embodiment, the present application provides an oral pharmaceutical composition containing 500 mg of metronidazole, wherein said composition provides Cₘₐₓ of not more than 9.0 μg/mL.

In another aspect, the present application provides an oral pharmaceutical composition comprising metronidazole, wherein the composition containing 500 mg of metronidazole is designed to provide a higher colonic intraluminal concentration while having a significantly lesser Cₘₐₓ, as compared to the Cₘₐₓ provided by commercially available dosage form i.e. FLAGYL® 500 mg tablets. The Cₘₐₓ of the composition of the present application observed not more than 9.0 μg/mL after administration, when tested in healthy human volunteers under fasting condition. The higher colonic intraluminal concentration provides equal or greater efficacy than FLAGYL® 500 mg tablets while the lesser Cₘₐₓ observed in the systemic circulation advantageously offers lesser side-effects as compared to the commercially available dosage form i.e. FLAGYL® 500 mg tablets.

In another embodiment, the present application provides an oral pharmaceutical composition containing 500 mg of metronidazole, wherein said composition provides AUC₀₋₄₈ of not more than 120 μg·h/mL.

In yet another aspect, the present application provides an oral pharmaceutical composition containing 500 mg of metronidazole, wherein the composition is designed to provide significantly lesser AUC₀₋₄₈, as compared to the AUC₀₋₄₈ provided by commercially available dosage form i.e. FLAGYL® 500 mg tablets. The AUC₀₋₄₈ of the composition of the present application observed not more than 120 μg·h/mL after administration, when tested in healthy human volunteers under fasting condition. The lesser AUC₀₋₄₈ observed in the systemic circulation advantageously offers lesser side-effects as compared to the commercially available dosage form i.e. FLAGYL® 500 mg tablets.

In another embodiment, the present application provides an oral pharmaceutical composition comprising metronidazole, wherein said composition provides Tₘₐₓ of not less than 4.0 hours.

In another aspect, the present application provides an oral pharmaceutical composition comprising metronidazole, wherein the composition is designed to provide significantly delayed Tₘₐₓ, as compared to the Tₘₐₓ provided by commercially available dosage form i.e. FLAGYL® 500 mg tablets. The Tₘₐₓ of the composition of the present application observed at not less than 4.0 hours after administration, when tested in healthy human volunteers under fasting condition. The delayed Tₘₐₓ of the present composition proves that the metronidazole is substantially released in the large intestine where the specific action of metronidazole is required. This advantageously ensures localized antibacterial activity, i.e., in colon and preserves the healthy proximal regions.

In another embodiment, the present application provides an oral pharmaceutical composition comprising metronidazole, wherein said composition provides T_{lag} of more than 1.0 hour.

Further aspect, of the present application provides an oral pharmaceutical composition comprising metronidazole, wherein the composition is designed to provide significantly delayed T_{lag}, as compared to the T_{lag} provided by commercially available dosage form i.e. FLAGYL® 500 mg tablets. The T_{lag} of the composition of the present application observed at more than 1.0 hour after administration, when tested in healthy human volunteers under fasting condition. The delayed T_{lag} of the present composition proves that the metronidazole is substantially released in the large intestine and ensuring greater concentration of metronidazole is delivered to the site i.e. colon.

In one aspect, the present application provides an oral pharmaceutical composition containing 500 mg of metronidazole, wherein said composition demonstrates Cₘₐₓ of not more than 9.0 μg/mL, when tested in healthy human volunteers under fasting condition.

In one aspect, the present application provides an oral pharmaceutical composition comprising metronidazole, wherein said composition demonstrates Tₘₐₓ of not less than 4.0 hours, when tested in healthy human volunteers under fasting condition.

In one aspect, the present application provides an oral pharmaceutical composition comprising metronidazole, wherein said composition T_{lag} of more than 1.0 hour, when tested in healthy human volunteers under fasting condition.

In one embodiment, the present application provides an oral pharmaceutical composition comprising metronidazole, wherein said composition demonstrates
Cₘₐₓ of not more than 9.0 μg/mL,
AUC₀₋₄₈ of not more than 120 μg·h/mL,
Tₘₐₓ of not less than 4.0 hours, and
T_{lag} of more than 1.0 hour,
when tested in healthy human volunteers under fasting condition.

In another embodiment, the present application provides an oral pharmaceutical composition comprising metronidazole, wherein said composition demonstrates
Tₘₐₓ of not less than 4.0 hours, and
T_{lag} of more than 1.0 hour,
   when tested in healthy human volunteers under fasting condition.

In another embodiment, the present application provides an oral pharmaceutical composition containing 500 mg of metronidazole, wherein said composition demonstrates
Cₘₐₓ of not more than 9.0 μg/mL,
AUC₀₋₄₈ of not more than 120 μg·h/mL.
   when tested in healthy human volunteers under fasting condition.

In another embodiment, an oral pharmaceutical composition comprising:
(i) a core containing metronidazole,
(ii) an optional separating layer,
(iii) a release layer comprising at least one release material which dissolves at a pH more than or equal to about 5.0, and
(iv) an optional protecting layer,
wherein said composition demonstrates Cₘₐₓ of not more than 9.0 μg/mL, when tested in healthy human volunteers under fasting condition.

In another embodiment, an oral pharmaceutical composition comprising:
(i) a core containing metronidazole,
(ii) an optional separating layer,
(iii) a release layer comprising at least one release material which dissolves at a pH more than or equal to about 5.0, and
(iv) an optional protecting layer,
wherein said composition demonstrates Tₘₐₓ of not less than 4.0 hours, when tested in healthy human volunteers under fasting condition.

In another embodiment, an oral pharmaceutical composition comprising:
(i) a core containing metronidazole,
(ii) an optional separating layer,
(iii) a release layer comprising at least one release material which dissolves at a pH more than or equal to about 5.0, and
(iv) an optional protecting layer,
wherein said composition demonstrates T_{lag} of more than 1.0 hour, when tested in healthy human volunteers under fasting condition.

In another embodiment, an oral pharmaceutical composition comprising:
(i) a core containing metronidazole,
(ii) an optional separating layer,
(iii) a release layer comprising at least one release material which dissolves at a pH more than or equal to about 5.0, and
(iv) an optional protecting layer,
wherein said composition demonstrates Tₘₐₓ of not less than 4.0 hours and T_{lag} of more than 1.0 hour, when tested in healthy human volunteers under fasting condition.

In another aspect, an oral pharmaceutical composition comprising:
(i) a core containing metronidazole,
(ii) an optional separating layer,
(iii) a release layer comprising at least one release material which dissolves at a pH more than or equal to about 5.0, and
(iv) an optional protecting layer,
wherein said composition demonstrates
   Cₘₐₓ of not more than 9.0 μg/mL,
   AUC₀₋₄₈ of not more than 120 μg·h/mL,
   Tₘₐₓ of not less than 4.0 hours, and
   T_{lag} of more than 1.0 hour,
when tested in healthy human volunteers under fasting condition.

In another embodiment, the present application provides an oral pharmaceutical composition comprising:
(i) a core containing metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5, and
(iv) an optional protecting layer,
wherein said composition demonstrates Cₘₐₓ of not more than 9.0 μg/mL, when tested in healthy human volunteers under fasting condition.

In another embodiment, the present application provides an oral pharmaceutical composition comprising:
(i) a core containing metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5, and
(iv) an optional protecting layer,
wherein said composition demonstrates Tₘₐₓ of not less than 4.0 hours, when tested in healthy human volunteers under fasting condition.

In another embodiment, the present application provides an oral pharmaceutical composition comprising:
(i) a core containing metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5, and
(iv) an optional protecting layer,
wherein said composition demonstrates T_{lag} of more than 1.0 hour, when tested in healthy human volunteers under fasting condition.

In another embodiment, the present application provides an oral pharmaceutical composition comprising:
(i) a core containing metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5, and
(iv) an optional protecting layer,
wherein said composition demonstrates Tₘₐₓ of not less than 4.0 hours and T_{lag} of more than 1.0 hour, when tested in healthy human volunteers under fasting condition.

In another embodiment, the present application provides an oral pharmaceutical composition comprising:
(i) a core containing metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5, and
(iv) an optional protecting layer,
wherein said composition demonstrates
   Cₘₐₓ of not more than 9.0 μg/mL,
   AUC₀₋₄₈ of not more than 120 μg·h/mL,
   Tₘₐₓ of not less than 4.0 hours, and
   T_{lag} of more than 1.0 hour,
when tested in healthy human volunteers under fasting condition.

In another embodiment, the present application provides an oral pharmaceutical composition comprising:
(i) a core comprising metronidazole,
(ii) a separating layer, and
(iii) a release layer comprising of:
a. at least one release material which dissolves at a pH more than or equal to about 5.0, and/or
b. at least one release material which dissolves at a pH more than or equal to about 6.5,
wherein said composition has
   Tₘₐₓ of not less than 4.0 hours, and
   T_{lag} of more than 1.0 hour,
when administered to a human subject under fasting condition.

In one aspect of the above embodiment, the composition containing 500 mg of metronidazole. The composition has
Cₘₐₓ of not more than 9.0 μg/mL,
AUC₀₋₄₈ of not more than 120 μg·h/mL,

In another embodiment, the present application provides an oral pharmaceutical composition comprising:
(i) a core comprising metronidazole,
(ii) a separating layer, and
(iii) a release layer comprising of:
   a. at least one release material which dissolves at a pH more than or equal to about 5.0, and/or
   b. at least one release material which dissolves at a pH more than or equal to about 6.5,
   wherein said composition has
      Cₘₐₓ of not more than 9.0 μg/mL,
      AUC₀₋₄₈ of not more than 120 μg·h/mL,
      Tₘₐₓ of not less than 4.0 hours, and
      T_{lag} of more than 1.0 hour,
when administered to a human subject under fasting condition.

In another aspect, the oral metronidazole composition according to the present application is capable of releasing the active ingredient (i.e., metronidazole) in the terminal ileum or cecum, thus ensuring localized antibacterial activity. As a result, the composition offers greater site-specificity and substantially reduces the systemic exposure of the metronidazole thereby reducing and/or eliminating any of the side effects observed with currently available metronidazole compositions i.e. FLAGYL® 500 mg tablets.

In one aspect, the efficacy of metronidazole, in the treatment of infections of the large intestine, and in particular of the colon, can be substantially potentiated without any undesired effects, by providing an oral pharmaceutical composition comprising metronidazole, wherein said composition demonstrates
Cₘₐₓ of not more than 9.0 μg/mL,
AUC₀₋₄₈ of not more than 120 μg·h/mL,
Tₘₐₓ of not less than 4.0 hours, and
T_{lag} of more than 1.0 hour,
when tested in healthy human volunteers under fasting condition.

Accordingly, the efficacy of metronidazole, in the treatment of infections of the large intestine, and in particular of the colon, can be substantially potentiated without any undesired effects, by providing an oral pharmaceutical composition comprising:
(i) a core containing metronidazole,
(ii) an optional separating layer,
(iii) a release layer comprising at least one release material which dissolves at a pH more than or equal to about 5.0, and
(iv) an optional protecting layer,
wherein said composition demonstrates
   Cₘₐₓ of not more than 9.0 μg/mL,
   AUC₀₋₄₈ of not more than 120 μg·h/mL,
   Tₘₐₓ of not less than 4.0 hours, and
   T_{lag} of more than 1.0 hour,
   when tested in healthy human volunteers under fasting condition.

Accordingly, the efficacy of metronidazole, in the treatment of infections of the large intestine, and in particular of the colon, can be substantially potentiated without any undesired effects, by providing an oral pharmaceutical composition comprising:
(i) a core containing metronidazole,
(ii) an optional separating layer,
(iii) a release layer comprising of:
   a. at least one release material which dissolves at a pH more than or equal to about 5.0, and/or
   b. at least one release material which dissolves at a pH more than or equal to about 6.5, and
(iv) an optional protecting layer,
wherein the said composition demonstrates
   Cₘₐₓ of not more than 9.0 μg/mL,
   AUC₀₋₄₈ of not more than 120 μg·h/mL,
   Tₘₐₓ of not less than 4.0 hours, and
   T_{lag} of more than 1.0 hour,
   when tested in healthy human volunteers under fasting condition.

In another embodiment, the composition releases metronidazole substantially in the large intestine upon administration to a patient in need of treatment with metronidazole.

In another embodiment, the composition releases metronidazole substantially in the distal portion of small intestine upon administration to a patient in need of treatment with metronidazole.

In another embodiment, the composition releases metronidazole substantially in the colon upon administration to a patient in need of treatment with metronidazole.

In one variant, the composition contains a core which comprises a matrix material and metronidazole, which are interspersed or dispersed in one another. In one variant, the matrix material may be a cellulose derivative or another substance suitable for erosion or diffusion release of the active ingredient.

The composition of the present application can be readily employed in a method for treating a patient infected with a microorganism which is susceptible to metronidazole, said method comprises administering to a patient having the infection a composition of the invention sufficient to eliminate or resolve an infection caused by the microorganisms.

In another embodiment, the composition provides a method of treating the intestinal infections, comprising administration to a patient in need thereof.

In another embodiment, the composition provides a method of treating the infections of large intestine, comprising administration to a patient in need thereof.

In another embodiment, the composition provides a method of treating the infections of distal portion of small intestine, comprising administration to a patient in need thereof.

In another embodiment, the composition provides a method of treating the infections of colon, comprising administration to a patient in need thereof.

The method of treating infections of the large intestine may additionally include other anti-infective or other pharmaceutical active agents.

In one aspect, there is provided a process for preparing an oral pharmaceutical composition, comprising:
(i) providing a core containing metronidazole,
(ii) optionally, applying a separating layer on the core,
(iii) applying a release layer comprising at least one release material which dissolves at a pH more than or equal to about 5.0, and
(iv) optionally, applying a protecting layer onto the release layer.

In another aspect, there is provided a process for preparing an oral pharmaceutical composition, comprising:
(i) providing a core containing metronidazole,
(ii) optionally, applying a separating layer on the core,
(iii) applying a release layer comprising of:
   a. at least one release material which dissolves at a pH more than or equal to about 5.0, and/or
   b. at least one release material which dissolves at a pH more than or equal to about 6.5, and
(iv) optionally, applying a protecting layer onto the release layer.

In another aspect, there is provided a process for preparing an oral pharmaceutical composition, comprising:
(i) providing a core containing metronidazole and at least one rate-controlling agent,
(ii) optionally, applying a separating layer on the core,
(iii) applying a release layer comprising at least one release material which dissolves at a pH more than or equal to about 5.0, and
(iv) optionally, applying a protecting layer onto the release layer.

In yet another aspect, there is provided a process for preparing an oral pharmaceutical composition, comprising:
(i) providing a core containing metronidazole and at least one rate-controlling agent,
(ii) optionally, applying a separating layer on the core,
(iii) applying a release layer, comprising of:
   a. at least one release material which dissolves at a pH more than or equal to about 5.0, and/or
   b. at least one release material which dissolves at a pH more than or equal to about 6.5, and
(iv) optionally, applying a protecting layer onto the release layer.

Generally the pharmaceutical compositions of the present application are prepared in unit dosage forms such as tablets, pills, capsules, powders, granules and/or microgranules.

The core containing the metronidazole of the instant composition can be prepared by any of the processes known in the art, such as by blending metronidazole with one or more pharmaceutically acceptable excipients using conventional methods, such as, simple granulation followed by sieving; extrusion and marumerization or spheronization; fluidized bed granulation; rotogranulation; pelletization; micropelletization, compression, etc.

In another aspect, the core containing metronidazole additionally may comprise at least one rate-controlling agent, to provide controlled release of the metronidazole. The rate-controlling agent can be blended with metronidazole and at least one pharmaceutically acceptable excipient or can be coated over the core containing metronidazole.

Metronidazole as used herein also encompasses pharmaceutically acceptable, pharmacologically active derivatives of metronidazole, their pharmaceutically acceptable salts, active metabolites of metronidazole and their pharmaceutically acceptable salts, unless otherwise noted. The solid state form of metronidazole used in the composition of the present application is not critical. For example, metronidazole can be amorphous or crystalline.

The term “pharmaceutically acceptable salts” as used herein includes those salts which are, within the scope of sound medical judgment, suitable for use in contact with the tissues of humans and lower animals without undue toxicity, irritation, allergic response and the like, which are well known in the art. The salts can be prepared in situ during the final isolation and purification of the compounds of the invention, or separately by reacting the pharmaceutically active substance having a free base function with a suitable organic acid or inorganic acid.

Examples of pharmaceutically acceptable nontoxic acid addition salts include, but not limited to, salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid lactobionic acid or malonic acid or by using other methods used in the art such as ion exchange techniques. Other pharmaceutically acceptable salts include, but not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartarate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

The term “release layer” denotes a physical feature of the formulation intended to effect release of the metronidazole from the core. The “release layer” herein includes all layers applied over the metronidazole core. For instance, the release layer also includes the separating layers and the protecting layers.

The term “release material” denotes a physical substance that facilitates the functioning of the release layer. Further the “release material” is intended to breakdown/erode/dissolve at pH more than or equal to about 5.0.

The recitation ‘releases metronidazole substantially in the large intestine” denotes the primary target site of release of the metronidazole in the gastrointestinal tract of the patient upon oral administration of the formulation. The recitation ‘releases metronidazole substantially in the distal portion of small intestine or in the colon” denotes the primary target site of release of the metronidazole in the gastrointestinal tract of the patient upon oral administration of the formulation. The release material should be non-toxic and is predominantly soluble in the intestinal fluid, but substantially insoluble in the gastric juices.

Suitable examples of release materials include, but are not limited to, polyvinyl acetate phthalate (PVAP), hydroxypropylmethyl-cellulose acetate succinate (HPMCAS), cellulose acetate phthalate (CAP), methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and commonly sold under the tradename of EUDRAGIT® L or EUDRAGIT® S, which are anionic copolymers based on methacrylate and available as powders) also commercially available as “methacrylic acid copolymer, type A” NF, “methacrylic acid copolymer, type B” NF, “methacrylic acid copolymer, type C” NF, methacrylic acid-methyl methacrylate copolymer, ethyl methacrylate-methylmethacrylate-chlorotrimethylammonium ethyl methacrylate copolymer, and the like, and combinations comprising one or more of the foregoing materials. Other examples include natural resins, such as shellac, SANDARAC®, copal collophorium, and combinations comprising one or more of the foregoing polymers. The release material of the composition can be combination of release materials that would dissolve in pH more than 5.0, for example, poly(methacrylic acid-co-ethyl acrylate) 1:1, available under the tradename “EUDRAGIT® L 30 D-55”, and the release material which dissolves in pH more than 6.5, for example, poly(methyl acrylate-co-methyl methacrylate-co-methacrylic acid) 7:3:1, available under the trade name “EUDRAGIT® FS 30 D”.

The release layer, separating layer and protecting layer may additionally contain one or more pharmaceutically acceptable plasticizers in order to obtain desirable mechanical properties, improve flexibility and strength of the coating. Example of plasticizers include, but not limited to, triacetin, citric acid esters, triethyl citrate, acetyl triethyl citrate, acetyl tributyl citrate, phthalic acid esters, diethyl phthalate, dibutyl sebacate, cetyl alcohol, polyethylene glycols, propylene glycol, triethylene glycol, polysorbates, oleic acid, ethyleneglycol monoleate, glyceryl monostearate, castor oil and mixtures thereof.

Along with the plasticizers, the release layer(s) may also have one or more pore-formers and anti-tacking materials. The pore-formers can be organic or inorganic, and include, but are not limited to, one or more hydrophilic polymers, such as starch, gums, alginates, polysaccharides, polyvinylprrolidone, polyethylene glycol, acrylic acid derivatives, and cellulose derivatives like hydroxypropyl cellulose, hydroxypropyl methylcellulose, hydroxyethylcellulose, hydroxymethylcellulose, carboxymethylcellulose, methylcellulose, sodium carboxy methylcellulose and mixtures thereof; polycarbonates comprised of linear polyesters of carbonic acid in which carbonate groups reoccur in the polymer chain. Alternatively, suitable pore formers include small molecules such as lactose or metal stearates, and combinations comprising one or more of the foregoing materials.

Anti-tacking materials are included to reduce film-to-film coefficient of friction, thereby reducing the associated problems i.e., strips, adhering to one another, etc. Examples of suitable anti-tacking materials include, but not limited to, talc, cornstarch, colloidal silicon dioxide, DL-Leucine, sodium lauryl sulfate, metallic stearates and combinations thereof.

In general, the separating layer is composed of coherent materials, such as polymeric materials, and/or finely powdered one or more solid excipients which constitute fillers. When a sugar is used in the separating layer, it is applied in the form of an aqueous solution and constitutes part of or the whole of the coherent material which sticks to the separating layer together. In addition to or instead of the sugar, a polymeric material may also be used in the separating layer.

For example, substances such as hydroxypropylmethylcellulose, polyvinylpyrrolidone, hydroxypropylcellulose and the like may be used in small amounts to increase the adherence and coherence of the separating layer. It is further advisable to use a filler excipient in the separating layer to increase the smoothness and to provide some rigidity to the layer. Substances such as finely powdered talc, silicon dioxide and the like are universally accepted as pharmaceutical excipients and may be added as is convenient in the circumstances to fill and smooth the separating layer.

The non-limiting functions of the separating layer maybe enumerated as follows: a) is to facilitate the application of the release layer, b) to prolong the metronidazole core's resistance to acid conditions, c) to improve stability by inhibiting any interaction between the drug and the release layer, and d) to improve stability, among various other functions. The smoothing function of the separating layer is purely mechanical, the objective of which is to improve the coverage of the release layer and to avoid thin spots in it, caused by bumps and irregularities on the core.

Generally, a protecting layer over the release layer is not necessary in every case, but frequently improves the elegance of the product and its handling, storage and machinability and may provide further benefits as well. The simplest protecting layer is simply a small amount of an anti-static ingredient such as talc or silicon dioxide, simply dusted on the surface of the pellets. Another simple protecting layer is a small amount of a wax such as beeswax melted onto the circulating mass of pellets to further smooth the pellets, reduce static charge, prevent any tendency for pellets to stick together, and increase the hydrophobicity of the surface.

Sometimes protecting layers may constitute a final sprayed-on layer of ingredients. For example, a thin layer of polymeric material such as hydroxypropylmethylcellulose, polyvinylpyrrolidone and the like may be applied. The polymeric material may also carry a suspension of an opacifier, a bulking agent such as talc, or a coloring material, particularly an opaque finely divided color agent such as red or yellow iron oxide.

All the layers, i.e. separating layer, release layer and the protecting layer, may be applied as solution/dispersion of coating ingredients using any conventional technique known in the art such as spray coating in a conventional coating pan or fluidized bed processor or dip coating.

Examples of solvents used for preparing a solution/dispersion of the coating ingredients include, but are not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran and mixtures thereof.

The core comprising metronidazole may additionally include at least one rate-controlling agent. The term “rate-controlling agent(s)” as used herein are meant to control/sustain the release rate of metronidazole. One or more rate-controlling agents can be blended with metronidazole and other excipients or can be coated over the metronidazole containing cores.

The “rate-controlling agent(s)” can be hydrophilic or hydrophobic or combinations of both. Suitable examples of hydrophilic rate-controlling agents include, but not limited to, cellulose derivatives such as hydroxypropylcellulose, hydroxypropylmethylcellulose, hydroxyethylcellulose, hydroxymethylcellulose, carboxymethylcellulose, methylcellulose, sodium carboxy methylcellulose or combinations thereof; polyvinylpyrrolidone, polyvinyl acetate, copolymer of vinylpyrrolidone and vinyl acetate, polysaccharides, polyalkylene glycols, starch, gums and derivatives; and combinations comprising one or more of the foregoing materials.

Suitable examples of hydrophobic rate-controlling agents include, but are not limited to, ethyl cellulose, cellulose acetate, cellulose acetate butyrate, hydroxypropyl methylcellulose phthalate, poly (alkyl) methacrylate, and copolymers of acrylic or methacrylic acid esters, waxes, shellac, zein, castor oil, hydrogenated vegetable oils and combinations comprising one or more of the foregoing materials.

The composition of the invention may contain suitable amounts of pharmaceutically acceptable excipients that would be necessary for preparing appropriate dosage forms. Examples of pharmaceutically acceptable excipients that can be used in the composition of the present invention include, but not limited to, one or more diluents, binders, disintegrants, lubricants/glidants, surfactants, buffers, wetting agents, coloring agents, flavoring agents or combinations thereof.

Examples of fillers or diluents include, but not limited to, corn starch, lactose, white sugar, sucrose, sugar compressible, sugar confectioners, glucose, sorbitol, calcium carbonate, calcium dihydrogen phosphate dihydrate, calcium phosphate-dibasic, calcium phosphate-tribasic, calcium sulfate, microcrystalline cellulose, silicified microcrystalline cellulose, cellulose powdered, dextrates, dextrins, dextrose, fructose, kaolin, lactitol, mannitol, starch, starch pregelatinized and mixtures thereof.

Examples of binders include, but not limited to, povidones, various starches known in the art, including corn starch, pregelatinized starch, microcrystalline celluloses (MCC), silicified MCC (e.g., PROSOLV™ HD 90), microfine celluloses, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropyl methylcellulose or hypromelloses (e.g., KLUCEL™ EF, METHOCEL™ E5 premium) and other pharmaceutically acceptable substances with cohesive properties.

Examples of disintegrants include, but not limited to, cross-linked polyvinyl pyrrolidone, corn starch, potato starch, maize starch and modified starches, agar-agar, calcium carbonate, sodium carbonate, alginic acids, cross-carmellose sodium, sodium starch glycolate, microcrystalline cellulose and mixtures thereof.

Examples of lubricants and glidants that can be used in the present invention include, but are not limited to, colloidal silicon dioxide, such as AEROSIL® 200, talc, stearic acid, magnesium stearate, calcium stearate, solid polyethylene glycols, sodium stearyl fumarate, silica gel and mixtures thereof and other substances with lubricating or gliding properties.

The term “surfactant” is used in its conventional sense in the present invention. Any surfactant is suitable, whether it is amphoteric, non-ionic, cationic or anionic. Examples of suitable surfactants include, but are not limited to, sodium lauryl sulfate, polysorbates such as polyoxyethylene sorbitan monooleate, monolaurate, monopalmitate, monostearate or another ester of polyoxyethylene sorbitan (e.g., the commercially available Tweens, sodium dioctylsulfosuccinate (DOSS), lecithin, stearyl alcohol, cetostearyl alcohol, cholesterol, polyoxyethylene ricin oil, polyoxyethylene fatty acid glycerides, poloxamers (e.g., PLURONICS F68® and Fl 08®, which are block copolymers of ethylene oxide and propylene oxide); polyoxyethylene castor oil derivatives or mixtures thereof.

Examples of buffers that can be used in the present invention include, but not limited to, phosphate, acetate, citrate, succinate and histidine buffers.

Examples of wetting agents that can be used in the present invention include, but not limited to, ammonium lauryl sulfate, sodium lauryl sulfate, and the like.

The coloring agents and flavoring agents of the present invention may be selected from any FDA approved colors and flavors for oral use.

The present invention is illustrated below by reference to the following example. However, one skilled in the art will appreciate that the specific methods and results discussed are merely illustrative of the invention, and not to be construed as limiting the invention.

### EXAMPLES

The compositions of the present example are prepared by following manufacturing process:

### Manufacturing Process-I:

1. Metronidazole, microcrystalline cellulose and a part of polyvinylpyrrolidone are mixed together.
2. Remaining part of polyvinylpyrrolidone is made into solution and granulated with mixture of step 1.
3. The granulated mass of step 2 is dried and milled to get fine granules of desired size.
4. The granules of step 3 are mixed with other excipients, i.e., colliodal silicon dioxide and magnesium stearate to form a uniform blend and further compressed into tablets
5. The tablets of step 4 are coated with a suspension made from separating layer ingredients to form separating layer
6. The tablets of step 5 are coated with a suspension made from given release layer ingredients to form release layer.

### Manufacturing Process-II:

1. Metronidazole, microcrystalline cellulose and a part of polyvinylpyrrolidone are mixed together.
2. Remaining part of polyvinylpyrrolidone is made into solution and granulated with mixture of step 1.
3. The granulated mass of step 2 is dried and milled to get fine granules of desired size.
4. The granules of step 3 are mixed with other excipients form a uniform blend and further compressed into tablets
5. The tablets of step 4 are coated with a suspension made from given rate-controlling agents.
6. The coated tablets of step 5 are again coated with a suspension made from given release layer ingredients to form release layer.

### Manufacturing Process-III:

1. Metronidazole, microcrystalline cellulose, lactose, colliodal silicon dioxide and a portion of polyvinylpyrrolidone are mixed together
2. Remaining part of polyvinylpyrrolidone is made into solution and granulated with mixture of step 1.
3. The granulated mass of step 2 is dried and milled to get fine granules of desired size.
4. The granules of step 3 are mixed with other excipients to form a uniform blend and further compressed into tablets.
5. The tablets of step 4 are coated with a suspension made from separating layer ingredients to form separating layer
6. The tablets of step 5 are coated with a suspension made from given release layer ingredients to form release layer.

### Examples 1-4

The compositions of the present examples are prepared by following manufacturing process-I:

| | Percent w/w | | | |
|---|---|---|---|---|
| | Exam- | | Exam- | Exam- |
| Ingredients | ple 1 | Example 2 | ple 3 | ple 4 |

Core:

| | | | | |
|---|---|---|---|---|
| Metronidazole | 61.3 | 60.5 | 61.3 | 60.1 |
| Microcrystalline cellulose | 13.5 | 13.3 | 13.5 | 13.2 |
| Polyvinylpyrrolidone | 6.2 | 6.0 | 6.2 | 6.0 |
| Water | q.s. | q.s. | q.s. | q.s. |
| Croscarmellose sodium | 6.1 | 6.0 | 2.5 | 2.4 |
| Hydroxypropylmethylcellulose | — | — | — | 4.2 |
| (METHOCEL ™ K100 LVCR) | | | | |
| Hydroxypropylmethylcellulose | — | — | 4.3 | — |
| (METHOCEL ™ E15 LV) | | | | |
| Colliodal silicon dioxide | 2.5 | 2.4 | 2.5 | 2.4 |
| Magnesium stearate | 0.6 | 0.6 | 0.6 | 0.6 |

Separating layer:

| | | | | |
|---|---|---|---|---|
| Hydroxypropylmethylcellulose | 1.9 | 1.9 | 1.9 | 1.9 |
| Polyethylene glycol 400 | 0.6 | 0.6 | 0.6 | 0.6 |
| Talc | 0.2 | 0.2 | 0.2 | 0.2 |

Release layer:

| | | | | |
|---|---|---|---|---|
| Methacrylic acid copolymer | 0.9 | 2.7 | 0.8 | 1.1 |
| (EUDRAGIT ® FS30D) | | | | |
| Methacrylic acid copolymer | 3.7 | 2.7 | 3.4 | 4.3 |
| (EUDRAGIT ® L30D-55) | | | | |
| Triethyl citrate | 0.2 | 0.3 | 0.2 | 0.3 |
| Talc | 2.3 | 2.7 | 2.1 | 2.7 |
| Water | q.s. | q.s. | q.s. | q.s. |

### Example-5

The compositions of the present examples are prepared by following manufacturing process-II:

| | |
|---|---|
| Ingredients | Percent (w/w) |

Core:

| | |
|---|---|
| Metronidazole | 61.70 |
| Microcrystalline cellulose | 13.58 |
| Polyvinylpyrrolidone | 6.17 |
| Water | q.s. |
| Croscarmellose sodium | 6.17 |
| Colliodal silicon dioxide | 2.47 |
| Magnesium stearate | 0.62 |

Rate-controlling layer:

| | |
|---|---|
| Ethyl cellulose | 2.86 |
| Hydroxypropylmethylcellulose | 1.54 |
| Dibutyl Sebacate (DBS) | 0.14 |
| Isopropyl alcohol | q.s. |
| Water | q.s. |

Release layer:

| | |
|---|---|
| Methacrylic acid copolymer | 1.54 |
| (EUDRAGIT ® FS30D) | |
| Methacrylic acid copolymer | 1.54 |
| (EUDRAGIT ® L30D-55) | |
| Triethyl citrate | 1.54 |
| Talc | 0.15 |
| Water | q.s. |

### Examples 6-12

The compositions of the present examples are prepared by following manufacturing process-III:

| | Percent w/w | | | | | | |
|---|---|---|---|---|---|---|---|
| Ingredients | Ex. 6 | Ex. 7 | Ex. 8 | Ex. 9 | Ex. 10 | Ex. 11 | Ex. 12 |

Core:

| | | | | | | | |
|---|---|---|---|---|---|---|---|
| Metronidazole | 62.06 | 60.90 | 62.06 | 62.05 | 62.05 | 62.06 | 62.15 |
| Microcrystalline | 6.21 | 6.09 | 6.21 | 6.21 | 6.21 | 6.21 | — |
| cellulose | | | | | | | |
| Lactose | — | — | — | — | — | — | 13.67 |
| Croscarmellose | 7.45 | 7.31 | 7.45 | 7.45 | 7.45 | 7.45 | — |
| sodium | | | | | | | |
| Colliodal silicon | 1.24 | 1.22 | 1.24 | 1.24 | 1.24 | 1.24 | — |
| dioxide | | | | | | | |
| Polyvinylpyrrolidone | 3.10 | 3.04 | 3.10 | 3.10 | 3.10 | 3.10 | 3.11 |
| Polyvinylpyrrolidone | 3.10 | 3.04 | 3.10 | 3.10 | 3.10 | 3.10 | 1.55 |
| Purified Water | q.s | q.s | q.s | q.s | q.s | q.s | q.s |
| Croscarmellose | 6.21 | 6.09 | 6.21 | 6.21 | 6.21 | 6.21 | — |
| sodium | | | | | | | |
| Colliodal silicon | 2.48 | 2.44 | 2.48 | 2.48 | 2.48 | 2.48 | 1.86 |
| dioxide | | | | | | | |
| Hydroxypropyl | — | — | — | — | — | — | 4.35 |
| methylcellulose | | | | | | | |
| Lactose | — | — | — | — | — | — | 3.42 |
| Magnesium stearate | 0.62 | 0.61 | 0.62 | 0.62 | 0.62 | 0.62 | 0.62 |

Separating layer:

| | | | | | | | |
|---|---|---|---|---|---|---|---|
| Hydroxypropyl | 1.98 | 1.94 | 1.98 | 1.98 | 1.98 | 1.98 | 1.94 |
| methylcellulose | | | | | | | |
| Talc | 0.59 | 0.58 | 0.59 | 0.59 | 0.59 | 0.59 | 0.58 |
| Polyethylene glycol | 0.20 | 0.19 | 0.20 | 0.20 | 0.20 | 0.20 | 0.19 |
| 400 | | | | | | | |

Release layer:

| | | | | | | | |
|---|---|---|---|---|---|---|---|
| Methacrylic acid | — | 3.85 | — | — | — | — | 3.85 |
| copolymer | | | | | | | |
| (EUDRAGIT ® L30D55) | | | | | | | |
| Methacrylic acid | 2.80 | — | — | — | — | — | — |
| copolymer | | | | | | | |
| (EUDRAGIT ® FS30D) | | | | | | | |
| Methacrylic acid | — | — | 2.80 | — | 0.84 | — | — |
| copolymer | | | | | | | |
| (EUDRAGIT ® L100) | | | | | | | |
| Methacrylic acid | — | — | — | 2.80 | 1.96 | — | — |
| copolymer | | | | | | | |
| (EUDRAGIT ® S100) | | | | | | | |
| Hydroxypropyl | — | — | — | — | — | 2.83 | — |
| methylcellulose | | | | | | | |
| acetate succinate | | | | | | | |
| Sodium lauryl | — | — | — | — | — | 0.09 | — |
| sulfate | | | | | | | |
| Talc | 1.40 | 1.92 | 1.40 | 1.40 | 1.40 | 0.85 | 1.93 |
| Triethyl Citrate | 0.56 | 0.77 | 0.56 | 0.56 | 0.56 | 0.99 | 0.77 |
| Water | q.s. | q.s. | q.s. | q.s. | q.s. | q.s. | q.s. |
| Isopropyl alcohol | — | — | q.s. | q.s. | q.s. | q.s. | — |

### Examples 13-16

The compositions of the present examples are prepared by following manufacturing process-III:

| | Percent w/w | | | |
|---|---|---|---|---|
| | Exam- | Example | Example | Example |
| Ingredients | ple 13 | 14 | 15 | 16 |

Core:

| | | | | |
|---|---|---|---|---|
| Metronidazole | 60.90 | 62.05 | 60.90 | 62.15 |
| Microcrystalline cellulose | 6.09 | 6.21 | 6.09 | 0.00 |
| Lactose | — | — | — | 17.09 |
| Polyvinylpyrrolidone | 6.09 | 6.21 | 6.09 | 4.66 |
| Croscarmellose sodium | 13.40 | 13.65 | 13.40 | 0.00 |
| Colliodal silicon dioxide | 3.65 | 3.72 | 3.65 | 1.86 |
| Hydroxypropyl | — | — | — | 4.35 |
| methylcellulose | | | | |
| (Hypromellose E15) | | | | |
| Magnesium stearate | 0.61 | 0.62 | 0.61 | 0.62 |

Separating layer:

| | | | | |
|---|---|---|---|---|
| Hydroxypropyl | 1.94 | 1.98 | 1.94 | 1.94 |
| methylcellulose | | | | |
| Talc | 0.58 | 0.59 | 0.58 | 0.58 |
| Polyethylene Glycol 400 | 0.19 | 0.20 | 0.19 | 0.19 |

Release layer:

| | | | | |
|---|---|---|---|---|
| Methacrylic acid copolymer | 0.84 | — | 0.77 | 0.84 |
| (EUDRAGIT ® FS30D) | | | | |
| Methacrylic acid copolymer | — | 2.80 | — | — |
| (EUDRAGIT ® L100) | | | | |
| Methacrylic acid copolymer | 3.38 | — | 3.08 | 3.38 |
| (EUDRAGIT ® L30D55) | | | | |
| Talc | 2.11 | 1.40 | 1.92 | 2.11 |
| Triethyl citrate | 0.21 | 0.56 | 0.77 | 0.21 |
| Water | q.s. | q.s. | q.s. | q.s. |
| Isopropyl alcohol | — | — | q.s. | q.s. |

### Examples 17-20

The compositions of the present examples are prepared by following manufacturing process-III:

| | Percent w/w | | | |
|---|---|---|---|---|
| | Example | Example | Example | Example |
| Ingredients | 17 | 18 | 19 | 20 |

Core:

| | | | | |
|---|---|---|---|---|
| Metronidazole | 62.41 | 62.41 | 63.58 | 62.41 |
| Microcrystalline | 13.73 | 13.73 | 13.99 | 13.73 |
| cellulose | | | | |
| Polyvinylpyrrolidone | 6.24 | 6.24 | 6.36 | 6.24 |
| Croscarmellose sodium | 6.24 | 6.24 | 6.36 | 6.24 |
| Colliodal silicon dioxide | 2.50 | 2.50 | 2.54 | 2.50 |
| Mg. Stearate | 0.62 | 0.62 | 0.64 | 0.62 |

Release layer:

| | | | | |
|---|---|---|---|---|
| Methacrylic acid | 1.07 | 2.66 | 2.95 | 3.73 |
| copolymer | | | | |
| (EUDRAGIT ® FS30D) | | | | |
| Methacrylic acid | 4.26 | 2.66 | 1.27 | 1.60 |
| copolymer | | | | |
| (EUDRAGIT ® | | | | |
| L30D55) | | | | |
| Talc | 2.67 | 2.66 | 2.11 | 2.67 |
| Triethyl citrate | 0.26 | 0.27 | 0.21 | 0.26 |
| Water | q.s. | q.s. | q.s. | q.s. |

### Examples 21-25

The compositions of the present examples are prepared by following manufacturing process-III:

| | Percent w/w | | | | |
|---|---|---|---|---|---|
| | Exam- | Exam- | Exam- | Exam- | Exam- |
| Ingredients | ple 21 | ple 22 | ple 23 | ple 24 | ple 25 |

Core:

| | | | | | |
|---|---|---|---|---|---|
| Metronidazole | 61.29 | 64.79 | 63.58 | 61.29 | 62.41 |
| Microcrystalline | 13.48 | 14.25 | 13.99 | 13.48 | 13.73 |
| cellulose | | | | | |
| Polyvinylpyrrolidone | 6.13 | 6.48 | 6.36 | 6.13 | 6.24 |
| Croscarmellose sodium | 6.13 | 6.48 | 6.36 | 6.13 | 6.24 |
| Colliodal silicon dioxide | 2.45 | 2.59 | 2.54 | 2.45 | 2.50 |
| Magnesium stearate | 0.61 | 0.65 | 0.64 | 0.61 | 0.62 |

Release layer:

| | | | | | |
|---|---|---|---|---|---|
| Methacrylic acid | 1.27 | 2.46 | 3.37 | 4.47 | 3.99 |
| copolymer | | | | | |
| (EUDRAGIT ® FS30D) | | | | | |
| Methacrylic acid | 5.11 | 0.61 | 0.84 | 1.91 | 1.34 |
| copolymer | | | | | |
| (EUDRAGIT ® | | | | | |
| L30D55) | | | | | |
| Talc | 3.20 | 1.54 | 2.11 | 3.20 | 2.67 |
| Triethyl citrate | 0.32 | 0.15 | 0.21 | 0.32 | 0.26 |
| Water | q.s. | q.s. | q.s. | q.s. | q.s. |

### Examples 26-31

The compositions of the present examples are prepared by following manufacturing process-III:

| | Percent w/w | | | | | |
|---|---|---|---|---|---|---|
| Ingredients | Ex. 26 | Ex. 27 | Ex. 28 | Ex. 29 | Ex. 30 | Ex. 31 |

Core:

| | | | | | | |
|---|---|---|---|---|---|---|
| Metronidazole | 60.33 | 62.05 | 62.05 | 62.06 | 60.90 | 59.51 |
| Microcrystalline | 6.03 | 6.21 | 6.21 | 6.21 | 6.09 | 5.95 |
| cellulose | | | | | | |
| Polyvinylpyrrolidone | 6.03 | 6.21 | 6.21 | 6.21 | 6.09 | 5.95 |
| Croscarmellose | 13.27 | 13.65 | 13.65 | 13.65 | 13.40 | 13.09 |
| sodium | | | | | | |
| Colliodal silicon | 3.62 | 3.72 | 3.72 | 3.72 | 3.65 | 3.57 |
| dioxide | | | | | | |
| Magnesium stearate | 0.60 | 0.62 | 0.62 | 0.62 | 0.61 | 0.60 |

Separating layer:

| | | | | | | |
|---|---|---|---|---|---|---|
| Hydroxypropyl | 1.93 | 1.98 | 1.98 | 1.98 | 1.94 | 1.90 |
| methylcellulose | | | | | | |
| Talc | 0.58 | 0.59 | 0.59 | 0.59 | 0.58 | 0.57 |
| Polyethylene Glycol | 0.19 | 0.20 | 0.20 | 0.20 | 0.19 | 0.19 |
| 400 | | | | | | |

Release layer:

| | | | | | | |
|---|---|---|---|---|---|---|
| Methacrylic acid | 2.39 | — | — | — | 1.92 | 2.80 |
| copolymer | | | | | | |
| (EUDRAGIT ® FS30D) | | | | | | |
| Methacrylic acid | — | 2.80 | 1.96 | — | — | — |
| copolymer | | | | | | |
| (EUDRAGIT ® S100) | | | | | | |
| Methacrylic acid | — | — | 0.84 | — | — | — |
| copolymer | | | | | | |
| (EUDRAGIT ® L100) | | | | | | |
| Methacrylic acid | 2.39 | — | — | — | 1.92 | 2.80 |
| copolymer | | | | | | |
| (EUDRAGIT ® L30D55) | | | | | | |
| Hydroxypropyl | — | — | — | 2.83 | — | — |
| methylcellulose | | | | | | |
| acetate succinate | | | | | | |
| (HPMCAS-HF) | | | | | | |
| Sodium lauryl | — | — | — | 0.09 | — | — |
| sulphate | | | | | | |
| Talc | 2.39 | 1.40 | 1.40 | 0.85 | 1.92 | 2.80 |
| Triethyl citrate | 0.24 | 0.56 | 0.56 | 0.99 | 0.77 | 0.28 |
| Water | q.s. | q.s. | q.s. | q.s. | q.s. | q.s. |
| Isopropyl alcohol | — | q.s. | q.s. | q.s. | — | — |

### In-Vitro Release Studies:

The in-vitro release studies for compositions of above examples were performed according to the USP dissolution test under one of the following test conditions:

### Example-32

Examples 1, 3, 4, 17, 18 & 20 were subjected to dissolution test for 120 minutes in 900 mL 0.1N HCl solution, 37° C. and 75 rpm; followed by 120 minutes in 900 mL solution buffered to a pH of 5.5, 37° C. and 75 rpm; and followed by 240 minutes in 900 mL solution buffered to a pH of 6.2, 37° C. and 75 rpm. Samples were withdrawn at designated time points, screened through a 10-micron filter only and analyzed for drug release by UV absorption. Regardless of the release layer composition, the compositions released substantially little or no drug in the 0.1 N HCl media and in the media buffered to a pH of 5.5. The average amount of drug released is shown in Table 1 below:

TABLE 1

| Dissolution | Percentage of drug released | | | | | | |
|---|---|---|---|---|---|---|---|
| media and | Example | Example | Example | Example | Example | Example | Example |
| Time | 1 | 3 | 4 | 17 | 18 | 19 | 20 |
| 0.1N HCl - | 0 | 0 | 0 | 0 | 0 | 0 | 0 |
| 120 min. | | | | | | | |
| pH 5.5 buffer - | 1 | 0 | 0 | 0 | 0 | 0 | 0 |
| 120 min. | | | | | | | |
| pH 6.2 buffer - | 0 | 0 | 0 | 10 | 0 | 0 | 0 |
| 30 min. | | | | | | | |
| pH 6.2 buffer - | 97 | 45 | 0 | 95 | 0 | 7 | 0 |
| 60 min. | | | | | | | |
| pH 6.2 buffer - | 100 | 99 | 3 | 97 | 0 | 57 | 0 |
| 90 min. | | | | | | | |
| pH 6.2 buffer - | — | — | 34 | 98 | 47 | 92 | 28 |
| 120 min. | | | | | | | |
| pH 6.2 buffer - | — | — | 71 | — | — | — | — |
| 180 min. | | | | | | | |
| pH 6.2 buffer - | — | — | 96 | — | — | — | — |
| 240 min. | | | | | | | |

### Example-33

Examples 13, 14, 15, 16 & 31 were subjected to dissolution test for 120 minutes in 900 mL 0.1N HCl solution, 37° C. and 75 rpm; followed by 60 minutes in 900 mL solution buffered to a pH of 5.5, 37° C. and 75 rpm; and followed by 180 minutes in 900 mL solution buffered to a pH of 6.5, 37° C. and 75 rpm. Samples were withdrawn at designated time points, screened through a 10-micron filter only and analyzed for drug release by UV absorption. Regardless of the release layer composition, the compositions released substantially little or no drug in the 0.1 N HCl media and in the media buffered to a pH of 5.5. The average amount of drug released is shown in Table 2 below:

TABLE 2

| Dissolution | Percentage of drug released | | | | |
|---|---|---|---|---|---|
| media and | Example | Example | Exam- | Example | Exam- |
| Time | 13 | 14 | ple 15 | 16 | ple 31 |
| 0.1N HCl - | 0 | 0 | 0 | 0 | 0 |
| 120 min. | | | | | |
| pH 5.5 buffer - | 0 | 0 | 0 | 0 | 0 |
| 120 min. | | | | | |
| pH 6.5 buffer - | 26 | 20 | 23 | 0 | 0 |
| 30 min. | | | | | |
| pH 6.5 buffer - | 95 | 97 | 100 | 8 | 26 |
| 60 min. | | | | | |
| pH 6.5 buffer - | 96 | 99 | 101 | — | 93 |
| 90 min. | | | | | |
| pH 6.5 buffer - | 96 | 99 | — | 69 | 98 |
| 120 min. | | | | | |
| pH 6.5 buffer - | — | — | — | 86 | — |
| 180 min. | | | | | |
| pH 6.5 buffer - | — | — | — | 99 | — |
| 180 min. | | | | | |

### Example-34

Example 21 was subjected to dissolution test for 120 minutes in 900 mL 0.1N HCl solution, 37° C. and 75 rpm; followed by 60 minutes in 900 mL acetate buffered solution to a pH of 4.5, 37° C. and 75 rpm; followed by 60 minutes in 900 mL solution buffered to a pH of 5.8, 37° C. and 75 rpm; and followed by 120 minutes in 900 mL solution buffered to a pH of 6.2, 37° C. and 75 rpm. Samples were withdrawn at designated time points, screened through a 10-micron filter only and analyzed for drug release by UV absorption. The average amount of drug released is shown in Table 3 below:

TABLE 3

| Dissolution media and | Percentage of drug released |
|---|---|
| Time | Example 21 |
| 0.1N HCl - 120 min. | 0 |
| pH 4.5 buffer - 60 min. | 0 |
| pH 5.8 buffer - 60 min. | 0 |
| pH 6.2 buffer - 30 min. | 0 |
| pH 6.2 buffer - 60 min. | 80 |
| pH 6.2 buffer - 90 min. | — |
| pH 6.2 buffer - 120 min. | 98 |

### Example-35

Examples 22 & 23 were subjected to dissolution test for 120 minutes in 900 mL 0.1N HCl solution, 37° C. and 75 rpm; followed by 120 minutes in 900 mL solution buffered to a pH of 6.5, 37° C. and 75 rpm; and followed by 120 minutes in 900 mL solution buffered to a pH of 7.0, 37° C. and 75 rpm. Samples were withdrawn at designated time points, screened through a 10-micron filter only and analyzed for drug release by UV absorption. The average amount of drug released is shown in Table 4 below:

TABLE 4

Percentage of drug released

| Dissolution media and Time | Example 22 | Example 23 |
|---|---|---|
| 0.1N HCl - 120 min. | 0 | 0 |
| pH 6.5 buffer - 120 min. | 5 | 0 |
| pH 7.0 buffer - 30 min. | 14 | 6 |
| pH 7.0 buffer - 60 min. | 25 | 27 |
| pH 7.0 buffer - 90 min. | 54 | 78 |
| pH 7.0 buffer - 120 min. | 73 | 89 |

### Example-36

Examples 2, 24 & 25 were subjected to dissolution test for 120 minutes in 900 mL 0.1N HCl solution, 37° C. and 75 rpm; followed by 120 minutes in 900 mL solution buffered to a pH of 6.0, 37° C. and 75 rpm; and followed by 120 minutes in 900 mL solution buffered to a pH of 6.2, 37° C. and 75 rpm. Samples were withdrawn at designated time points, screened through a 10-micron filter only and analyzed for drug release by UV absorption. The average amount of drug released is shown in Table 5 below:

TABLE 5

| | Percentage of drug released | | |
|---|---|---|---|
| Dissolution media and Time | Example 2 | Example 24 | Example 25 |
| 0.1N HCl - 120 min. | 0 | 0 | 0 |
| pH 6.0 buffer - 120 min. | 0 | 0 | 0 |
| pH 7.2 buffer - 30 min. | 15 | 0 | 0 |
| pH 7.2 buffer - 60 min. | 88 | 15 | 72 |
| pH 7.2 buffer - 90 min. | 96 | 96 | 98 |
| pH 7.2 buffer - 120 min. | 98 | 98 | 99 |

### Example-37

Examples 26-31 were subjected to dissolution test for 120 minutes in 900 mL 0.1N HCl solution, 37° C. and 75 rpm; followed by 60 minutes in 900 mL solution buffered to a pH of 6.0, 37° C. and 75 rpm; and followed by 120 minutes in 900 mL solution buffered to a pH of 7.5, 37° C. and 75 rpm. Samples were withdrawn at designated time points, screened through a 10-micron filter only and analyzed for drug release by UV absorption. Regardless of the release layer composition, the compositions released substantially no drug in the 0.1 N HCl media and in the media buffered to a pH of 6.0. The average amount of drug released is shown in Table 6 below:

TABLE 6

| Dissolution | Percentage of drug released | | | | | |
|---|---|---|---|---|---|---|
| media and | Example | Example | Example | Example | Example | Example |
| Time | 26 | 27 | 28 | 29 | 30 | 31 |
| 0.1N HCl - | 0 | 0 | 0 | 0 | 0 | 0 |
| 120 min. | | | | | | |
| pH 6.0 buffer - | 0 | 0 | 0 | 0 | 0 | 0 |
| 60 min. | | | | | | |
| pH 7.5 buffer - | 11 | 16 | 87 | 99 | 95 | 25 |
| 30 min. | | | | | | |
| pH 7.5 buffer - | 75 | 94 | 99 | 100 | 96 | 97 |
| 60 min. | | | | | | |
| pH 7.5 buffer - | 95 | 98 | 101 | — | 97 | 100 |
| 90 min. | | | | | | |
| pH 7.5 buffer - | 98 | 98 | — | — | 97 | 101 |
| 120 min. | | | | | | |

### Example 38

### In-Vivo Pharmacokinetics Study

The compositions of Examples 13, 16 and 26 (all the compositions containing 500 mg of metronidazole) are subjected to pharmacokinetic studies, in healthy, adult male subjects under fasting conditions. A randomized, open label, four way crossover study was performed. The examples were compared with FLAGYL® 500 mg tablets of G.D. Searle LLC, Division of Pfizer Inc. The results are shown in Table 7.

TABLE 7

| | | | | FLAGYL ® |
|---|---|---|---|---|
| Parameters | Example 13 | Example 16 | Example 26 | 500 mg |
| Cₘₐₓ (μg/mL) | 6.50 ± 2.44 | 6.83 ± 1.61 | 5.99 ± 2.05 | 11.11 ± 1.83 |
| AUC₀₋₄₈ | 99.16 ± 29.23 | 106.87 ± 27.49 | 94.71 ± 32.12 | 130.66 ± 30.33 |
| (μg · h/mL) | | | | |
| AUC_{0-∞} | 103.48 ± 33.20 | 111.08 ± 29.93 | 100.11 ± 36.64 | 133.86 ± 32.20 |
| (μg · h/mL) | | | | |
| Tₘₐₓ (h) | 9.75 | 9.00 | 11.75 | 1.50 |
| AUC₀₋₄₈/ | 0.97 ± 0.02 | 0.96 ± 0.02 | 0.95 ± 0.03 | 0.98 ± 0.01 |
| AUC_{0-∞} | | | | |
| Kₑₗ (1/h) | 0.09 ± 0.01 | 0.09 ± 0.01 | 0.09 ± 0.02 | 0.09 ± 0.02 |
| T_{lag} (h) | 2.50 | 2.00 | 3.50 | 0.00 |
| t_{1/2} (h) | 8.14 ± 1.57 | 8.33 ± 1.51 | 8.25 ± 1.72 | 8.09 ± 1.60 |

### Example-39

### Measurement of Coating Thickness

The compositions of Examples 13 and 31 were subjected to SEM (scanning electron microscopy) analysis as representative samples. The coating thicknesses of samples were measured using SEM model: Hitachi S-3400N. The intact tablets were cut using thin blade and attached to sample stubs using double side tapes. The samples were viewed using an accelerating voltage of 15 kilovolt at the magnification of 100× to 750×. The coating thickness was measured and the images were stored. The measured mean readings (no of tablets—6) are shown in Table 8:

TABLE 8

| | Mean coating |
|---|---|
| Example | thickness (μm) |
| Example-13 | 105.0 |
| Example-31 | 140.6 |

### Example 40

### Stability Studies

Accelerated stability study was conducted for Examples 13 and 26 at 40° C.±2° C. in 75%±5% relative humidity. The study was carried out for 3 months period of time. The measured mean parameters (no of tablets: 6) are shown in Table 9 and 10 respectively.

TABLE 9

| | | | In-vitro drug release | |
|---|---|---|---|---|
| | | Total | | Percentage |
| | | related | Dissolution media and | of |
| Period | Assay | substances | Time | drug released |
| Initial | 99.4% | 0.03% | 0.1N HCl - 120 min. | 0 |
| | | | pH 5.5 buffer - 60 min. | 0 |
| | | | pH 6.5 buffer - 90 min. | 96 |
| 1 month | 98.9% | 0.03% | 0.1N HCl - 120 min. | 0 |
| | | | pH 5.5 buffer - 60 min. | 0 |
| | | | pH 6.5 buffer - 90 min. | 99 |
| 2 months | 98.6% | 0.04% | 0.1N HCl - 120 min. | 0 |
| | | | pH 5.5 buffer - 60 min. | 0 |
| | | | pH 6.5 buffer - 90 min. | 101 |
| 3 months | 99.5% | 0.04% | 0.1N HCl - 120 min. | 0 |
| | | | pH 5.5 buffer - 60 min. | 0 |
| | | | pH 6.5 buffer - 90 min. | 99 |

TABLE 10

| | | | In-vitro drug release | |
|---|---|---|---|---|
| | | Total | | Percentage |
| | | related | Dissolution media and | of |
| Period | Assay | substances | Time | drug released |
| Initial | 100.7% | 0.03% | 0.1N HCl - 120 min. | 0 |
| | | | pH 6.0 buffer - 60 min. | 0 |
| | | | pH 7.5 buffer - 90 min. | 95 |
| 1 month | 100.7% | 0.03% | 0.1N HCl - 120 min. | 0 |
| | | | pH 6.0 buffer - 60 min. | 0 |
| | | | pH 7.5 buffer - 90 min. | 97 |
| 2 months | 99.3% | 0.04% | 0.1N HCl - 120 min. | 0 |
| | | | pH 6.0 buffer - 60 min. | 0 |
| | | | pH 7.5 buffer - 90 min. | 100 |
| 3 months | 98.6% | 0.04% | 0.1N HCl - 120 min. | 0 |
| | | | pH 6.0 buffer - 60 min. | 0 |
| | | | pH 7.5 buffer - 90 min. | 100 |

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

1. A metronidazole composition comprising:
(i) a core containing metronidazole, and
(ii) a release layer comprising at least one release material which dissolves/erodes in the aqueous environment at a pH of more than about 5.0.

2. The composition of claim 1, wherein the release layer comprises at least one additional release material which dissolves in the aqueous environment at a pH of more than or equal to about 6.5.

3. The composition of claim 1, wherein the composition comprises a separating layer disposed between said core and said release layer.

4. The composition of claim 1, wherein the composition comprises a protecting layer placed over the release layer.

5. The composition of claim 1, wherein the thickness of the release layer ranges from about 60 μm to about 180 μm.

6. The composition of claim 1, wherein the ratio of the release layer to the core of said composition ranges from about 4.0:1.0 to about 4.80:0.20, by weight.

7. The composition of claim 1, wherein the release layer of the composition comprises from about 1% w/w to about 20% w/w of the composition.

8. The composition of claim 1, wherein the release material(s) selected from the group comprising of polyvinyl acetate phthalate (PVAP), hydroxypropylmethyl-cellulose acetate succinate (HPMCAS), cellulose acetate phthalate (CAP), methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and commonly sold under the tradename of EUDRAGIT® L or EUDRAGIT® S, which are anionic copolymers based on methacrylate and available as powders) also commercially available as “methacrylic acid copolymer, type A” NF, “methacrylic acid copolymer, type B” NF, “methacrylic acid copolymer, type C” NF, methacrylic acid-methyl methacrylate copolymer, ethyl methacrylate-methylmethacrylate-chlorotrimethylammonium ethyl methacrylate copolymer, and combinations comprising one or more of the foregoing materials.

9. The composition of claim 1, wherein the release layer dissolves/erodes within the pH range of about 5.5 to about 7.5.

10. The composition of claim 1, wherein the composition comprises at least one rate-controlling agent.

11. The composition of claim 10, wherein the rate-controlling agent amount is in the range of 0 to about 40% of the total weight of the composition.

12. The composition of claim 11, wherein the rate-controlling agent is selected from the group comprising of hydroxypropylcellulose, hydroxypropylmethylcellulose, hydroxyethyl cellulose, hydroxymethylcellulose, carboxymethylcellulose, methylcellulose, sodium carboxy methylcellulose or combinations thereof; polyvinylpyrrolidone, polyvinyl acetate, copolymer of vinylpyrrolidone and vinyl acetate, polysaccharides, polyalkylene glycols, starch, gums and derivatives; ethyl cellulose, cellulose acetate, cellulose acetate butyrate, hydroxypropyl methylcellulose phthalate, poly (alkyl) methacrylate, and copolymers of acrylic or methacrylic acid esters, waxes, shellac, zein, castor oil, hydrogenated vegetable oils and combinations comprising one or more of the foregoing materials.

13. An oral pharmaceutical composition of metronidazole, wherein said composition releases not more than 10% of metronidazole at a period of 120 minute in 900 ml of 0.1N HCl solution and not less than 60% of metronidazole at a period of 240 minutes in 900 ml of aqueous buffer having pH of 6.5, when tested in a USP Type 2 apparatus at 75 rpm and 37° C.

14. The composition of claim 13, wherein the composition releases not less than 70% of metronidazole at a period of 240 minutes in 900 ml of aqueous buffer having pH of 6.5, when tested in a USP Type 2 apparatus at 75 rpm and 37° C.

15. (canceled)

16. (canceled)

17. The composition of claim 1, wherein the composition has T_{lag} of more than 1.0 hour, when administered to a human subject under fasting condition.

18. The composition of claim 1, wherein the composition has Cₘₐₓ of not more than 9.0 μg/mL, when administered to a human subject under fasting condition.

19. The composition of claim 1, wherein the composition has AUC₀₋₄₈ of not more than 120 μg·h/mL, when administered to a human subject under fasting condition.

20. The composition of claim 1, wherein the composition has Tₘₐₓ of not less than 4.0 hours, when administered to a human subject under fasting condition.

21. The composition of claim 1, wherein the composition has
Cₘₐₓ of not more than 9.0 μg/mL,
AUC₀₋₄₈ of not more than 120 μg·h/mL,
Tₘₐₓ of not less than 4.0 hours, and
T_{lag} of more than 1.0 hour,
when administered to a human subject under fasting condition.

22. An oral pharmaceutical composition comprising
(i) a core comprising metronidazole,
(ii) a separating layer, and
(iii) a release layer comprising of:
a. at least one release material which dissolves at a pH more than or equal to about 5.0, and/or
b. at least one release material which dissolves at a pH more than or equal to about 6.5;
wherein said composition has
Tₘₐₓ of not less than 4.0 hours, and
T_{lag} of more than 1.0 hour,
when administered to a human subject under fasting condition.

23. A method of treating intestinal infections comprising administration to a patient in need thereof the pharmaceutical composition of claim 1.

24. The method of claim 23, wherein the intestinal infections are infections of large intestine.

25. The method of claim 23, wherein the intestinal infections are infections of distal portion of small intestine.

26. The method of claim 23, wherein the intestinal infections are infections of colon.

27.-39. (canceled)

40. The composition of claim 22, wherein the composition contains 500 mg of metronidazole.

41.-43. (canceled)
